(19) United States
(12) Patent Application Publication
(10) Pub. No.: US 2018/0369715 A1
(43) Pub. Date: Dec. 27, 2018

(54) EXTRACTION DEVICE AND EXTRACTION METHOD FOR EXTRACTION OF CANNABIS

(71) Applicant: Wernard Emest Bruining, Tiel (NL)

(72) Inventor: Wernard Emest Bruining, Tiel (NL)

(21) Appl. No.: 15/895,185

(22) Filed: Feb. 13, 2018

Related U.S. Application Data
(63) Continuation of application No. PCT/NL2016/050576, filed on Aug. 15, 2016.

(30) Foreign Application Priority Data
Jun. 13, 2015 (NL) 2015304

(51) Int. Cl. B01D 11/02; A61K 36/185

(52) U.S. Cl. CPC B01D 11/0219; A61K 2236/33; A61K 36/185

(57) ABSTRACT

The invention relates to an extraction device for bringing cannabis into contact with an extraction fluid, wherein the device comprises a column provided with a bottom, and near the bottom with a continuous hole through which the extraction fluid is able to flow out of the column under the influence of gravity. In that way for instance oil can be extracted from cannabis.

### BACKGROUND OF THE INVENTION

The invention relates to an extraction device and extraction method for the treatment of cannabis.

Extraction techniques for the treatment of cannabis are known in the art. Usable substances (compounds or compositions) are extracted from cannabis by way of these dedicated methods and devices, for instance using alcohol (ethanol) or other suitable solvents.

Among others government authorities have developed methods, in particular for the analysis of cannabis in terms of quality.

According to the abstract, U.S. Pat. No. 3,715,189 describes a method for qualitative analysis for cannabis material, wherein a test liquid is held in a frangible inner container held in an outer container. The inner container has a frangible section that can be broken so that the material to be tested in the outer container gets into contact with the test liquid.

U.S. Pat. No. 6,403,126 relates to a method for extraction of cannabinoids, cannflavins and/or essential oils from hemp. Dried hemp is put into contact with ethanol, toluene or hexane for a period of time while being stirred. The liquid is filtered. The liquid is then for instance subjected to a chromatography for separating specific compounds.

U.S. Pat. No. 8,445,034 describes an assembly or method for preparing a medicine from cannabis plant material. Cannabis material is put into contact with an extraction fluid for a period of time. A reflux process or an ultrasonic process is being utilized.

US2003017216 describes a method for the preparation of a cannabis extract. A solvent is allowed to contact the cannabis for a period of time. The solvent is then evaporated.

US2013079531 describes rapid extraction of active ingredients from a herbal material using a cold solvent and a short mixing period. The solvent is below minus 10° C. The herbal material may be cannabis. The solvent may be a hydrocarbon such as hexane or pentane, but ethanol is preferred. The contact period is less than a few minutes.

WO2014159688 describes concentrates from cannabis. Many substances to be extracted and solvents are mentioned. The material is put into contact with extraction fluid for a period of time while being agitated.

Various methods to extract useful compounds or compositions from cannabis are furthermore known. For instance, in known methods a quantity of cannabis is put in a container, for instance a bucket. An excess of extraction fluid is poured over it. After some time, the extraction fluid is decanted and a quantity of extraction fluid is applied again. After repeating this several times, the collected extraction fluid is subjected to a distillation step wherein (a large part of) the extraction fluid is removed. Such a method requires a large excess of extraction fluid.

According to the abstract, CA2402033 describes “a method and device for extracting oils from plant material, of the type used for aroma therapy or cooking and the like. The device comprises a container having an inlet aperture in a top end and a plurality of outlet apertures in a bottom end opposite the inlet aperture. The plant material is supported in the container and another suitable receptacle. An organic solvent having a boiling point below ambient temperature, such as butane, is dispensed from a pressurized vessel into the inlet aperture of the container and is allowed to drain into the receptacle via the outlet apertures after forming a solution with the oils of the plant material. The organic solvent is then permitted to evaporate from the solution in the receptacle leaving the extracted oils in the receptacle.”

In the abstract, US2011/0133120 describes that “the invention provides for plant oil-extraction”. The abstract continues rather insignificantly with “It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims.”

According to the abstract, US2013/0251824 describes “A process and apparatus for extracting cannabinoids from cannabis plant material [sic] and for transporting low concentrations of cannabinoids in a solution of solvent includes at least a solvent tank, a hopper and one or more pumps. The plant material may be soaked, washed, and/or rinsed with a solvent that extracts essential elements from cannabis plant matter. The process at least includes filling a hopper with plant matter, moving solvent from a solvent tank to the hopper and moving solvent back to the solvent tank after an extraction. The best mode of the invention extracts cannabinoids and/or cannabis related essential elements from the cannabis plant matter and stores it in a solvent bath in a low density per unit volume of the solvent.”

According to the abstract, US2010/0130762 describes “compositions and methods for extracting oils comprising contacting material containing the oil with a composition comprising 1-chloro-3,3,3-trifluoropropene in an amount sufficient to dissolve at least a portion of the oil, and separating the solvent/oil solution from the material.”

The preparation of cannabis oil is shown in a video by Rick Simson, https://www.youtube.com/watch?v=KZXGH6mYr3Y, in which a bucket is filled with cannabis, onto which alcohol is poured and decanted several times, and the alcohol is evaporated.

The suggested methods are either complex or unsuitable to be used e.g. at home, enabling a patient to prepare a simple medicinal extract, in particular an extract from cannabis.

### SUMMARY OF THE INVENTION

It is an object of the invention to provide either an extraction device or a method that is easier in terms of construction and use.

For that purpose, the invention provides an extraction device for bringing cannabis into contact with an extraction fluid, wherein the device comprises a column provided with a bottom, and near the bottom with a continuous hole through which the extraction fluid is able to flow out of the column under the influence of gravity.

The invention further relates to a method for extracting cannabis using the extraction device according to any one of the preceding claims, comprising filling the column with a weighed quantity of cannabis, adding a predetermined quantity of extraction fluid in the column, collecting the extraction fluid flowing out of the opening, in a container.

In one embodiment the continuous hole has a diameter of between 1 and 5 mm. In particular the hole has a diameter of 1-2 mm. Due to the diameter of the hole it can be prevented that solids to be extracted are taken along with the extraction fluid leaving the column via the continuous hole.

In one embodiment the column has a diameter of 1-5 cm. Especially in combination with the dimensions of the hole, this proved to enable proper extraction.

In one embodiment the column has been provided with a calibration for a quantity of fluid.

In one embodiment the column has been provided with a stand for keeping the column at a set height.

In one embodiment, the column is a glass tube. It may have a diameter of 1-4 cm and be approximately 10-50 cm high.

The extraction fluid may be an extraction fluid known to experts. Particularly suitable are solvents or extraction agents for the extraction of elements from cannabis. More in particular the extraction fluids arc suitable for extracting oil-like elements. For instance, alcohol, in particular a 95% vol. ethanol, proved to be suitable. The hole can be relatively small then, as a result of which the solids will be left behind. In one embodiment of the method the extraction fluid is evaporated after the extraction.

In one embodiment a layer of cannabis is formed in the column. On top of that a pillar or column of ethanol is formed. At the bottom side near the cannabis, a high concentration of dissolved substance is formed, and at the top of the ethanol pillar the concentration will be lower down to practically zero. When operational the column of ethanol will sink through the layer of cannabis. Due to the ethanol's low viscosity it will also be able to leave the column through the small holes under the influence of gravity. Moreover, ethanol, contrary to a solvent such as for instance water, will sink properly through the layer of cannabis without being absorbed by the cannabis.

In one embodiment the cylindrical container with a continuous hole in the bottom is provided with a quantity of grams of cannabis, and a quantity of 5-7 times the weight in cannabis of 95% vol. ethanol is poured in. The ethanol of the extract is evaporated. In particular 5.5-6.5 times the weight in cannabis of 95% vol. ethanol is poured in. After evaporating the ethanol, cannabis oil is left.

The type of cannabis may be “recreational” cannabis or “industrial” cannabis such as for instance cultivated near the city of Groningen (the Netherlands). It may be the tips or fallen leaves, the quality of the oil is the same as is the extraction process. When using recreational cannabis, the oil harvested contains a lot of THC and little CBD and when using industrial hemp, the resulting oil contains little TI-IC and a lot of CBD.

One embodiment relates to “cannabis oil” diluted with a vegetable oil, such as for instance olive oil. One embodiment is diluted approximately 2×-5× (on mass basis). That means 1-4× the weight in vegetable oil relative to pure “cannabis oil” is being added. This will enable accurate dosage per drop and getting “high” can be avoided. The remedy can thus also be used for sensitive patients and children. Pure “cannabis oil” is recommended per drop the size of a rice grain, but in the event that the rice grain turns out slightly bigger than usual, the result is being “high” for hours. The use of “cannabis oil” that has only been diluted 3× is for instance recommended for cancer patients, simply because they need to use a lot.

The column is made of a solvent-resistant material. The column is particularly made of a material that is resistant to alcohol/ethanol. In one embodiment, the column is a graduated measuring cylinder.

In one embodiment the column is for instance made of glass. A 2-3 mm pin made of tungsten can for instance be used to make a hole in the bottom by not pressing the pin fully through the bottom. The hole made will then have a diameter of approximately 1-1.5 mm.

Alternatively, a solvent-resistant synthetic material could be used. A hole could be melted or drilled into it.

The invention further relates to a “kit-of-parts” for the extraction of cannabis, comprising a column as described.

The invention furthermore relates to an extraction device, or parts thereof provided with one or more of the characterizing measures described in the attached description and/or shown in the attached drawings.

It will be clear that the various aspects mentioned in this patent application can be combined and that each may individually qualify for a divisional patent application.

### BRIEF DESCRIPTION OF THE FIGURES

The attached FIGURE shows an embodiment of an extraction device.

The FIGURE is not necessarily to scale.

### DESCRIPTION OF THE EMBODIMENTS

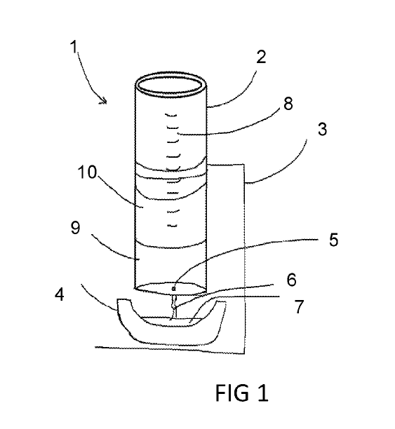

FIG. 1 shown an extraction device 1. The extraction device comprises a column 2 for containing the solid substance that needs to be extracted. The column 2 has been provided with a hole 5 near a lowest point. The column is held by means of a stand 3 above a collection container 4. The column has been provided with a calibration 8.

When in use, the column 2 is filled with the solid substance, indicated with reference number 9, from which one or more components need to be extracted. The weight of the solid substance has been determined beforehand. Based on said weight a quantity of solvent has subsequently been determined. The quantity of solvent, in this case indicated with reference number 10, is subsequently poured into the column 2. The solvent 10 runs through the solid substance 9 and via hole 5 runs in a trickle 6 or in drops and is collected in the collection container 4 in a quantity 7. The collection container can be provided with a heating element with which (a part of) the solvent 10 can be evaporated.

### EXAMPLE

Method for producing a standard 10 cc bottle of 5× diluted “cannabis oil” using the arrangement of FIG. 1. The hole selected here is 1.5 mm, the column is a 100 ml glass graduated measuring cylinder having a single hole in the bottom.
Clean properly dried cannabis, remove branches etc.
Reduce the volume by pulverizing or rubbing it through a sieve.
Weigh a quantity, for instance 20 gram, of cannabis and put it in the tube/column.
Press down gently from time to time, for instance using the handle of a ladle.
Pour an overall quantity of 120 ml of alcohol (80-95% ethanol) in the course of a number of times, and allow it to drain dry for an hour or so.
Allow the alcohol to evaporate in the evaporator. For instance, in a baby bottle heater.
Once all bubbles have disappeared for instance 2.5 cc of pure cannabis oil will be left behind, then add 10 cc of olive oil and stir well for 5-10 minutes.
Pour the 5× (volume) diluted “cannabis oil” into for instance a 10 cc bottle with a pipette.

The entire process takes a few hours and always provides the same quality of product.

Such a product is highly identical in terms of strength and as a consequence has a predictable effect. The oil also proves to contain less resin, as a result of which it can be avoided that the cannabis oil becomes turbid.

A column as described above, may have an adapted height and diameter. The column may for instance be over a meter high, and have a diameter of up to 30 cm. Larger set-ups, such as a few meters high and a diameter of 50 cm or more are possible. In practice, in case of a cylindrical column the height will be at least 10 times the diameter. Such a column may have been provided with a single hole of for instance 1-2 mm. In one embodiment the bottom has been provided with a number of such holes. By providing the column with a layer of dried cannabis, and arranging a column of ethanol on top of it and allowing the column of ethanol to pass through the column of cannabis, controlled extraction of cannabis oil takes place. A ratio as mentioned above will yield an optimal result.

It will be clear that the above description is included to illustrate the operation of preferred embodiments of the invention and not to limit the scope of the invention. Starting from the above explanation many variations that fall within the spirit and scope of the present invention will be evident to an expert.

### LIST OF REFERENCE NUMBERS

1 extraction device
2 column
3 stand
4 collection container
5 efflux opening
6 effusing extraction fluid
7 extraction fluid in collection container
8 calibration
9 solid substance to be extracted, such as cannabis
10 extraction fluid/solvent

1. Extraction device for bringing cannabis into contact with an extraction fluid, wherein the device comprises a column provided with a bottom, and near or in the bottom with a continuous hole through which the extraction fluid is able to flow out of the column under the influence of gravity.

2. Extraction device according to claim 1, wherein the continuous hole has a diameter of between 1 and 5 mm, in particular 1-2 mm.

3. Extraction device according to claim 1, wherein the column has a diameter of 1-5 cm.

4. Extraction device according to any one of the preceding claims, wherein the column has been provided with a stand for keeping the column at a set height.

5. Method for extracting cannabis using the extraction device according to any one of the preceding claims, comprising filling the column with a weighed quantity of cannabis, adding a predetermined quantity of extraction fluid in the column, collecting the extraction fluid flowing out of the opening, in a container.

6. Method according to claim 6, wherein ethanol is being used as extraction fluid, wherein in particular per gram of dried cannabis between 4 and 7 gram of ethanol is added.

7. Kit-of-parts for the extraction of cannabis oil, comprising a tubular column having a diameter of 1-4 cm, in the bottom provided with a hole having a diameter of 1-2 mm.

8. Kit-of-parts according to claim 7, further provided with a collection device underneath the hole, and a heating device in contact with the collection device.
